(19) United States
(12) Patent Application Publication
(10) Pub. No.: US 2015/0149090 A1
(43) Pub. Date: May 28, 2015

(54) LIVING PLANT MONITORING SYSTEMS

(71) Applicant: Living Systems, Inc., Houston, TX (US)

(72) Inventors: Federico T. MARQUEZ, Houston, TX (US); Mark SULLIVAN, Houston, TX (US)

(21) Appl. No.: 14/552,217

(22) Filed: Nov. 24, 2014

Related U.S. Application Data
(60) Provisional application No. 61/907,922, filed on Nov. 22, 2013.

(51) Int. Cl. G01D 7/00; G01D 7/12; G01N 33/00

(52) U.S. Cl. CPC G01D 7/00; G01N 33/0098; G01D 7/12
USPC 702/2

(57) ABSTRACT

A plant monitoring system for the monitoring of environmental parameters that affect growing plants and that is capable of producing audible and visible alerts when the health of the plant is threatened. In some embodiments, the plant monitoring system is configured to produce calibrated alerts specific to the type of plant being monitored based upon information stored in an on-line database. In still other embodiments, plants are remotely monitored by web-enabled devices which further allow for additional calibration settings to be entered by the user.

### CROSS-REFERENCE TO RELATED APPLICATION

This application claims the benefit of U.S. Provisional Application No. 61/907,922, filed Nov. 22, 2013, the contents of which is incorporated herein by reference in its entirety.

### FIELD OF THE TECHNOLOGY

The present application relates generally to a system for the monitoring of environmental parameters that affect growing plants. More specifically, embodiments of the present disclosure relate to a plant monitoring system that communicates environmental parameters, such as soil moisture, to a computer or web-enabled device. In even more specific embodiments, the disclosure relates to a plant monitoring system capable of sending alerts or notifications to a computer or web-enabled device and/or to the remote sensing unit itself, based upon specific information stored in an on-line database.

### BACKGROUND OF THE TECHNOLOGY

While many people are interested in growing plants, maintaining plant health is often elusive due to uncertainty as to how to meet the specific needs of the particular plant. For example, plants frequently suffer from being either over-watered or under-watered. Therefore a monitoring system capable of alerting a caregiver as to the appropriate time to water a plant is desirable. Further, a plant monitoring system capable of monitoring the sum of environmental parameters necessary for optimal growth and alerting the caregiver when one or more plant need is not being met is desirable.

### BRIEF DESCRIPTION OF THE DRAWINGS

Implementations of the present application will now be described, by way of example only, with reference to the attached figures, wherein:

FIG. 1 is a schematic view of the plant monitoring system according to one embodiment of the invention, where at least one battery-operated sensing device 100 is in wireless communication with a receiver unit 101 which is in further communication with a computer or web-enabled device 102.

FIG. 2 is a schematic view of the plant monitoring system according to one embodiment of the invention, where at least one battery-operated sensing device 100 is in wireless communication with a receiver unit 101 which is in further communication with a server 104 through an internet router 103, the server 104 being capable of sending notifications or alerts to a computer or web-enabled device 102.

FIG. 3 is a schematic view of the plant monitoring system according to one embodiment of the invention, where at least one battery-operated sensing device 100 is in wireless communication with a receiver unit 101 which is in further communication with a server 104 through an internet router 103, the server 104 being in further communication with a database 105 and being capable of sending notifications or alerts to a computer or web-enabled device 102 based upon data stored in the database 105.

FIG. 4 is a perspective view of a remote moisture sensing device according to one embodiment of the invention. The remote sensing device is comprised of a micro-controller, two 1.5V button cell batteries, and related sensors that are housed in a thin, lightweight plastic housing 111 with two tinned or nickel plated electrodes that penetrate the soil or plant growth medium. The device may be used to measured soil moisture levels manually using a button 110 on the device which forces a reading. This allows the device to be used to measure soil moisture levels for multiple plants by quickly inserting the device into the soil of different plants and taking a forced reading.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the implementations described herein. However, the implementations described herein can be practiced without these specific details. In other instances, methods, procedures and components have not been described in detail so as not to obscure the related relevant function being described. Also, the description is not to be considered as limiting the scope of the implementations described herein. It will be understood that descriptions and characterizations of embodiments set forth in this disclosure are not to be considered as mutually exclusive, unless otherwise noted.

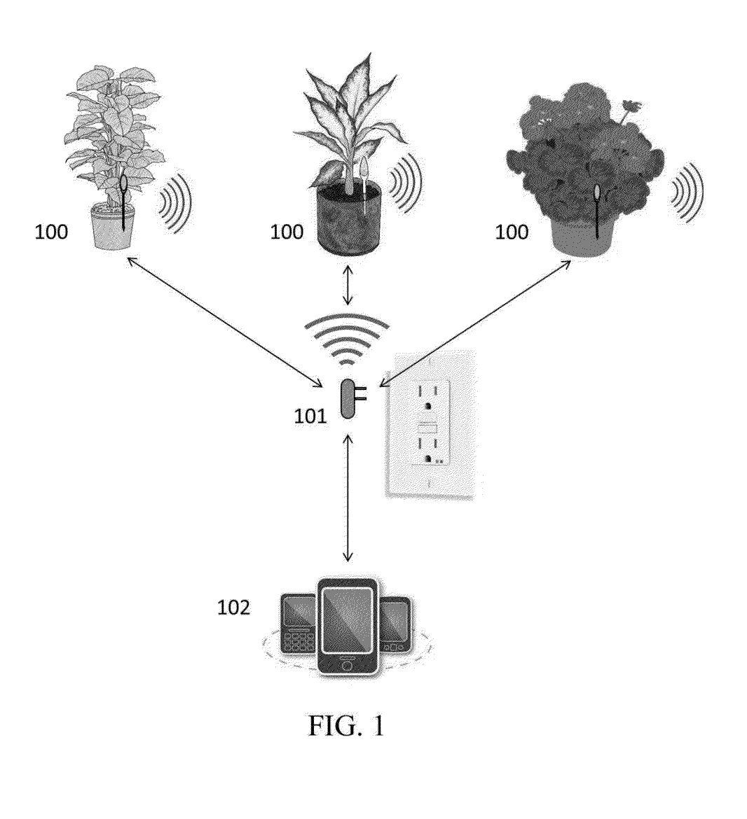

The present disclosure relates to a plant monitoring system for the monitoring of environmental parameters necessary for the optimal growth of living plants. FIG. 1 is a schematic view of a first embodiment of the plant monitoring system. The plant monitoring system consists of one or more battery-operated sensing devices 100 capable of measuring at least one environmental parameter in a plant growth medium, such as a soil. The sensing device is in wireless communication with a receiver unit 101 that is capable of communicating environmental data, alerts or notifications to a computer or other web-enabled device 102 such as a mobile phone, personal digital assistant (PDA), or tablet computer. The receiver unit 101 may optionally operate as a remote hub and may be plugged into a normal wall socket. In one embodiment, the sensing device 100 transmits data via radio frequency to the receiver unit 101. The receiver unit 101 can be configured to collect information from up to 50 separate and unique sensing devices 100. Optionally, the web-enabled device 102 may be used to monitor the environmental parameters necessary for proper plant maintenance and growth using a mobile application that is configured to accept notifications as well as to accept user input that may be used to ensure the health of specific plant types.

The sensing device may be further configured to provide visible and audible alerts if an environmental parameter is unfavorable to plant health. For example, the sensing device may provide an alert if moisture levels in the growth medium are too low and plant health is threatened. The sensing device may be further configured to contain a photosensor which disables the audible alerts when the lights are off in order to not disturb plant caregivers when they may be sleeping. In some embodiments, the sensing device measures soil moisture. In other embodiments, the sensing device measures light levels, temperature, and/or soil pH, either in conjunction with soil moisture measurements or independent of soil moisture measurements.

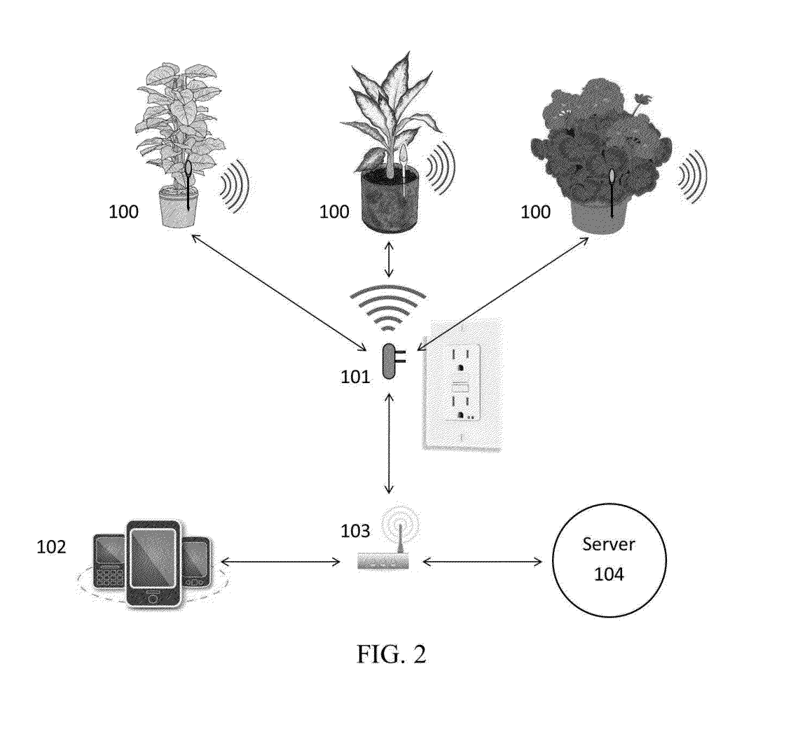

FIG. 2 is a schematic view of a second embodiment of the plant monitoring system. The plant monitoring system consists of one or more battery-operated sensing devices 100 capable of measuring at least one environmental parameter in a plant growth medium, such as a soil. The sensing device is in wireless communication with a receiver unit 101 that is in communication with a server 104 through an internet router 103. The server 104 is further configured to communicate environmental data, alerts or notifications to a computer or other web-enabled device 102 such as a mobile phone, personal digital assistant (PDA), or tablet computer. The server 104 is also capable of issuing alerts or notifications to the sensing device 100 itself. Optionally, the web-enabled device 102 may be used to monitor the environmental parameters necessary for proper plant maintenance and growth using a mobile application that is configured to accept notifications as well as to accept user input that may be used to ensure the health of specific plant types.

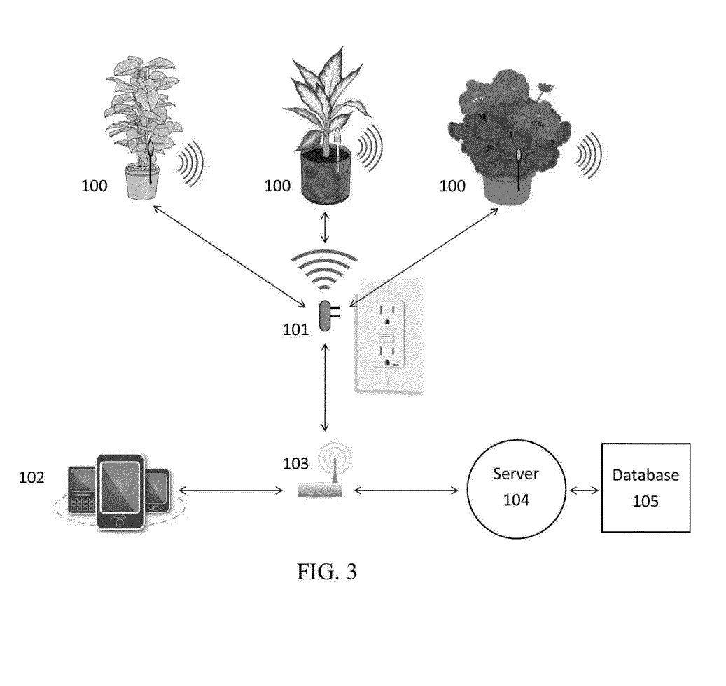

FIG. 3 is a schematic view of a third embodiment of the plant monitoring system. The plant monitoring system consists of one or more battery-operated sensing devices 100 capable of measuring at least one environmental parameter in a plant growth medium, such as a soil. The sensing device is in wireless communication with a receiver unit 101 that is in communication with a server 104 through an internet router 103. The server 104 is configured to be in communication with an on-line database 105 and further configured to issue alerts or notifications based upon data contained in the database. The server may also communicate environmental data, alerts or notifications to a computer or other web-enabled device 102 such as a mobile phone, personal digital assistant (PDA), or tablet computer. The server 104 is also capable of issuing alerts or notifications to the sensing device 100 itself. Additionally, the sensing device 100 may be further configured to send environmental data, such as moisture, light, and temperature levels, to a server which stores the data on an on-line database 105. In one embodiment, the database 105 is linked to a website. The information stored in the database 105 may be used to calibrate alert settings based on specific plant type and issue notifications or monitoring information to caregivers to ensure optimum plant health. Other parameters, such as soil type, may also be stored in the database 105 in order to calibrate settings necessary to maintain optimum plant health. Optionally, the web-enabled 102 device may be used to monitor the environmental parameters necessary for proper plant maintenance and growth using a mobile application that is configured to accept notifications as well as to accept user input that may be used to ensure the health of specific plant types.

In one embodiment, the sensing device is a soil moisture sensing device and the plant monitoring system is a soil moisture monitoring system. The soil moisture monitoring system consists of a device for the alerting of low moisture content in a soil that is comprised of a micro-controller, one or more batteries, a sensing device capable of measuring soil moisture, one or more speakers capable of producing an audible alert, and one or more light emitting diodes (LEDs) or other warning beacons capable of producing a visible alert. The device is further configured to be in wireless communication with a receiver unit that is in further communication with a server capable of issuing alerts or notifications to a computer or web-enabled device. The server may optionally be in communication with an on-line database and further configured to issue notifications based upon data contained in the database. The device may be further configured to be calibrated based on plant type and related settings in the on-line database. In still other embodiments, the device may contain additional sensors for the monitoring of light levels, temperature, and soil pH.

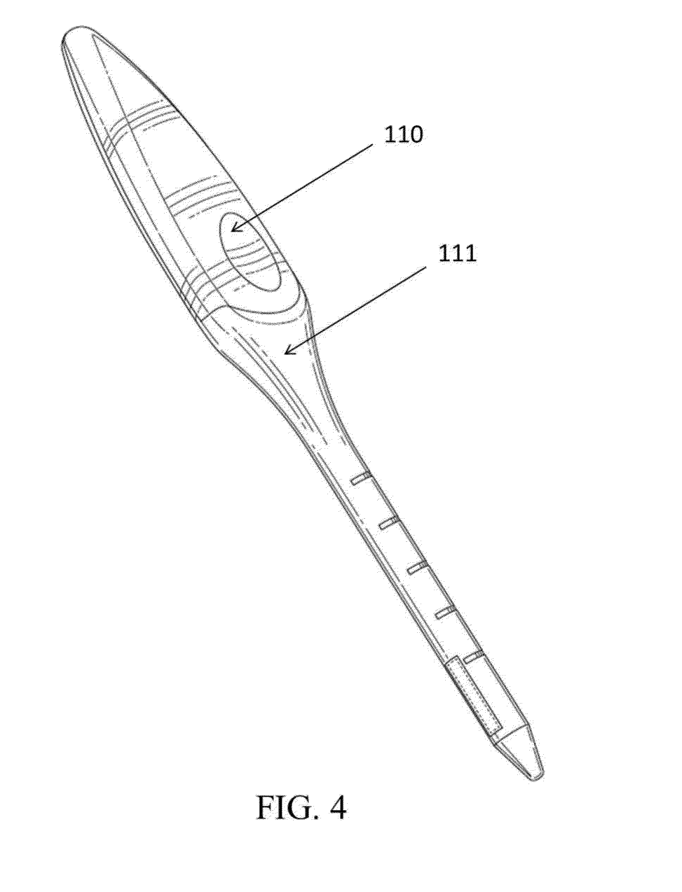

In one embodiment, the soil moisture monitoring system is comprised of a remote soil moisture sensing device. FIG. 4 is a perspective view of a remote moisture sensing device according to one embodiment. The remote sensing device is comprised of a micro-controller, two 1.5V button cell batteries, and related sensors that are housed in a thin, lightweight plastic housing 111 with two tinned or nickel plated electrodes that penetrate the soil or plant growth medium. The device senses soil moisture using an impedance method known in the art. The impedance setting is programmed into the microprocessor. If moisture levels in the growth medium or soil drop too low, the remote device produces an audible alert via a speaker, piezo device or other means. Simultaneously, the device produces a visible alert using a LED or other warning beacon. The tinned or nickel plated electrodes will not corrode when placed in the soil or growth medium of the container, allowing for continuous monitoring of plant health. In other embodiments, the electrodes may be plated with other materials known in the art in order to prevent corrosion.

The remote moisture sensing device can be placed in any type of plant container regardless of container, soil type or plant type. In one embodiment, the device accurately measures soil moisture levels either remotely or manually using a button 110 on the device which forces a reading. This allows the device to be used for continuous measurement of a single plant in a plant container or to measure soil moisture for multiple plants by quickly inserting the device into the soil of different plants and taking a forced reading.

In one embodiment, the remote plant monitoring device is configured for extended battery life using a programming technique that repurposes a watchdog timer to only allow the processor to run intermittently when a new measurement is taken. A watchdog timer is a common feature in computers and processors used in control applications. The original purpose of the watchdog timer is to reset the processor if there is a hardware problem or program crash that prohibits proper operation. The watchdog timer typically has a timeout value of a few hundred milliseconds. The properly operating software program resets the watchdog timer periodically. If the program fails to reset the timer before the timeout, as would be expected if the program crashes or gets stuck in a loop, then the timer goes off and resets the system. Commonly, the watchdog timer utilizes a timebase that is separate from the main processor clock so that the watchdog timer may continue to function and reset the processor if there is a problem with the main clock. This independent timekeeping allows the watchdog timer to be repurposed through a programming technique so as to greatly extend the battery life of the plant monitoring device. Specifically, the watchdog timer is set for the longest possible time interval. For example, on a PIC12F510 micro-controller, the watchdog timer may be set for 2.3 seconds. The main clock oscillator of the processor may be further stopped by a program. After 2.3 seconds elapses the watchdog timer resets the processor, the main clock restarts and the program starts running again. Since the remote sensing device only needs to take a measurement intermittently, a counter in the firmware program can be used to keep track of the number of watchdog restarts and after a certain number of restarts, the sensing device takes a measurement. For example, if the watchdog timer is set for 2.3 seconds and the sensing device is configured to take a measurement every 10 minutes, the device would take a reading after every 256 restarts. Since the process of updating the counter and halting the processor again takes a few tens of microseconds, and the process of taking a reading and flashing a green LED takes about 10 milliseconds, the system only draws full power from the batteries for about 10 milliseconds out of 10 minutes or 1.7E−5 of the time. For example, the battery life of the remote device may be greatly extended from months to years through use of this programming technique.

In another embodiment, the plant monitoring system allows for the remote monitoring of plants while the caregiver is away and may be coupled to a plant watering system to allow for remote watering of the plants.

In at least one embodiment, the device includes nickel plated probes or electrodes that enable the device's sensor to stay in soil for indefinite time without risk of corrosion. The device can also include a push button feature that allows for a forced reading of some or all of the sensors. This provides ability to use this device as a monitoring device to take readings of multiple plants at once. The device can be configured to read light levels and be programmed to not give audible alarm if light is too low. This can prevent the device from waking someone up at night if it senses that moisture levels are too low.

What is claimed is:

1. A plant monitoring system comprising:
at least one battery-operated sensing device 100 capable of measuring at least one environmental parameter in a plant growth medium;
a receiver unit 101 having wireless communication with the at least one sensing device 100;
at least one computer or web-enabled device 102 capable of receiving notifications or data from the receiver unit.

2. The plant monitoring system of claim 1, further comprising a server 104 configured to be in communication with the at least one sensing device 100 and the receiver unit 101 through an internet router 103 and further configured to issue alerts or notifications to the sensing device 100 or computer or web-enabled device 102.

3. The plant monitoring system of claim 2, wherein the server 104 is in communication with an on-line database 105 and is configured to issue notifications based upon data contained in the database 105.

4. The plant monitoring system of claim 3, wherein the sensing device 100 is further comprised of an alert system capable of producing a visible or audible alert.

5. The plant monitoring system of claim 4, wherein the environmental parameter measured is soil moisture.

6. The plant monitoring system of claim 4, wherein the environmental parameter measured is light level.

7. The plant monitoring system of claim 4, wherein the environmental parameter measured is temperature.

8. The plant monitoring system of claim 4, wherein the environmental parameter measured is soil pH.

9. A soil moisture monitoring system comprising:
at least one battery-operated soil moisture sensing device 100;
a receiver unit 101 having wireless communication with the at least one soil moisture sensing device 100;
at least one computer or web-enabled device 102 capable of receiving notifications or data from the server 104.

10. The soil moisture monitoring system of claim 9, further comprising a server 104 configured to be in communication with the at least one soil moisture sensing device 100 and the receiver unit 101 through an internet router 103 and further configured to issue alerts or notifications to the soil moisture sensing device 100 or web-enabled device 102.

11. The soil moisture monitoring system of claim 10, wherein the server 104 is in communication with an on-line database 105 and further configured to issue notifications based upon data contained in the database 105.

12. The soil moisture monitoring system of claim 11, wherein the soil moisture sensing device 100 is further comprised of an alert system capable of producing a visible or audible alert.

13. Device for the alerting of low moisture content in a soil comprising:
a micro-controller;
at least one battery;
at least one sensing device capable of measuring soil moisture;
at least one speaker capable of producing an audible alert;
at least one light emitting diode (LED) or other beacon capable of producing a visible alert;
wherein the device for the alerting of low moisture content is configured to be in wireless communication with a receiver unit 101 that is in further communication with a server 104 capable of issuing alerts or notifications to a computer or web-enabled device 102.

14. The device according to claim 13, wherein the server 104 is in communication with an on-line database 105 and further configured to issue notifications based upon data contained in the database 105.

15. The device according to claim 14, further comprising a light sensor capable of deactivating the audible alerts when the lights are off.

16. The device according to claim 15, wherein the device for the alerting of low moisture content is further configured for extended battery life by intermittent running of the processor as controlled by a specifically configured watchdog timer.

17. The device according to claim 16, further comprising a sensor capable of measuring light level.

18. The device according to claim 17, further comprising a sensor capable of measuring temperature.

19. The device according to claim 18, further comprising a sensor capable of measuring soil pH.

20. The device according to claim 16, further configured to be calibrated based on plant type and related settings in the on-line database 105.

21. The device according to claim 1, wherein the device includes nickel plates sensors or electrodes.

22. The device according to claim 1, wherein the device is configured to enables the device to receive a signal to take a forced reading.

23. The device according to claim 1, further comprising an ambient light sensor and wherein the device is configured to not alert when ambient light sensed is below a predetermined threshold.
